(19) United States
(12) Patent Application Publication
(10) Pub. No.: US 2011/0131840 A1
(43) Pub. Date: Jun. 9, 2011

(54) AFFECTING FOOT POSITION

(76) Inventor: Stanley W. YANG, Upland, CA (US)

(21) Appl. No.: 12/633,706

(22) Filed: Dec. 8, 2009

(51) Int. Cl. A61F 5/14

(52) U.S. Cl. 36/144

(57) ABSTRACT

Systems and methods for affecting a position of a foot are provided. In some aspects, a foot orthotic system comprises a compartment system configured to engage a shoe and engage at least a portion of a foot when the foot resides in or on the shoe. The compartment system may be adjustable from a first shape to a second shape. The foot orthotic system may also comprise one or more elongate members. In some aspects, as the compartment system is adjusted from the first shape to the second shape, an elongate member may change from a first state to a second state that is more taut than is the first state. In some aspects, when the compartment system engages at least a portion of the foot and is adjusted from the first shape to the second shape, a position of the foot relative to the shoe is adjusted.

### FIELD

The present invention generally relates to foot orthotics, and more particularly to systems and methods for affecting a position of a foot.

### BACKGROUND

Pronation of a foot is a rotation of the medial bones in the midtarsal region of the foot inward and downward so that in walking the foot tends to come down on its inner margin. Pronation occurs when the arch flattens out as the foot strikes the ground. Normally, the foot may pronate to absorb shock when the heel hits the ground, and to assist in balance during mid-stance. The ankle may tip towards the inside. Excessive pronation can be problematic because the shifting causes increased stress on the inside/medial aspect of the foot. It can pull on the stabilizing muscles in the lower leg (e.g., posterior tibialis) and can cause the knee to shift to the inside. This action is associated with foot pain as well as knee pain, shin splints, achilles tendinitis, posterior tibial tendinitis, Piriformis syndrome, and plantar fasciitis.

Supination of the foot is the opposite motion of pronation. A foot is in supination when the ankle appears to be tipped to the outside so that a person with supination may appear to be standing on the outside border of the foot. Excessive supination may predispose the ankle to injury because the stabilizing muscles on the outside of the lower leg (e.g., peroneals) are in a stretched position, potentially causing ligament damage.

### SUMMARY

According to various aspects of the subject disclosure, a foot orthotic system for affecting a position of a foot is provided. The foot orthotic system may comprise a compartment system configured to engage a shoe and engage at least a portion of a foot when the foot resides in or on the shoe. The compartment system may be adjustable from a first shape to a second shape. The foot orthotic system may also comprise one or more elongate members. Each of the one or more elongate members may have a first portion, a second portion, and a body therebetween. The first portion may be coupled to at least one of a first part of the compartment system and a first part of one of the one or more elongate members. The second portion may be coupled to at least one of a second part of the compartment system and a second part of one of the one or more elongate members. In some aspects, as the compartment system is adjusted from the first shape to the second shape, at least one of the one or more elongate members changes from a first state to a second state that is more taut than is the first state. In some aspects, when the compartment system engages at least a portion of the foot and is adjusted from the first shape to the second shape, a position of the foot relative to the shoe is adjusted. In some aspects, the foot orthotic system may also comprise the shoe.

According to certain aspects, the compartment system may comprise a first line extending from at least one of (a) a medial edge of the shoe at a toe area of the shoe, and (b) a lateral edge of the shoe at the toe area, to at least one of (a) the medial edge at a heel area of the shoe, and (b) the lateral edge at the heel area. The medial edge may extend from an end of the shoe at the toe area to an end of the shoe at the heel area. The lateral edge may extend from the end of the shoe at the toe area to the end of the shoe at the heel area. The compartment system may also comprise a second line extending from the medial edge to the lateral edge. The second line may be substantially perpendicular to a longitudinal axis of the shoe. The longitudinal axis of the shoe may extend from the end of the shoe at the toe area to the end of the shoe at the heel area. The compartment system may also comprise a third line extending from the medial edge to the lateral edge. The third line may be substantially perpendicular to the longitudinal axis of the shoe and disposed between the second line and the heel area. The compartment system may also comprise a fourth line extending from the medial edge to the lateral edge. The fourth line may be substantially perpendicular to the longitudinal axis of the shoe and disposed between the third line and the heel area.

In some aspects, the compartment system may comprise a first medial compartment being adjustable from an unexpanded shape to an expanded shape. The first medial compartment may have a foot engaging portion that is substantially bounded by the first line, the second line, the third line, and the medial edge, when the first medial compartment is in the expanded shape. The compartment system may also comprise a first lateral compartment being adjustable from an unexpanded shape to an expanded shape. The first lateral compartment may have a foot engaging portion that is substantially bounded by the first line, the second line, the third line, and the lateral edge, when the first lateral compartment is in the expanded shape.

In some aspects, the compartment system may also comprise a second medial compartment being adjustable from an unexpanded shape to an expanded shape. The second medial compartment may have a foot engaging portion that is substantially bounded by the first line, the third line, the fourth line, and the medial edge, when the second medial compartment is in the expanded shape. The compartment system may also comprise a second lateral compartment being adjustable from an unexpanded shape to an expanded shape. The second lateral compartment may have a foot engaging portion that is substantially bounded by the first line, the third line, the fourth line, and the lateral edge, when the second lateral compartment is in the expanded shape.

In some aspects, the compartment system may also comprise a third medial compartment being adjustable from an unexpanded shape to an expanded shape. The third medial compartment may have a foot engaging portion that is substantially bounded by the first line, the fourth line, and at least one of the medial edge at the heel area and the lateral edge at the heel area, when the third medial compartment is in the expanded shape. The compartment system may also comprise a third lateral compartment being adjustable from an unexpanded shape to an expanded shape. The third lateral compartment may have a foot engaging portion that is substantially bounded by the first line, the fourth line, and at least one of the lateral edge at the heel area and the medial edge at the heel area, when the third lateral compartment is in the expanded shape.

In certain aspects, as measured along a length of the shoe, a distance between the end of the shoe at the toe area and the second line, a distance between the second line and the third line, a distance between the third line and the fourth line, and a distance between the fourth line and the end of the shoe at the heel area are substantially equal. In some aspects, the second line may be spaced such that between about 20% and about 35% of a length of the shoe lies between the end of the shoe at the toe area and the second line. The third line may be spaced such that between about 45% and about 55% of a length of the shoe lies between the end of the shoe at the toe area and the third line. The fourth line may be spaced such that between about 70% and about 85% of a length of the shoe lies between the end of the shoe at the toe area and the fourth line.

In some aspects, the respective foot engaging portions of the first medial compartment, the first lateral compartment, the second medial compartment, the second lateral compartment, the third medial compartment, and the third lateral compartment are configured to engage at least 70% of an under side of the foot. In some aspects, the first line may be substantially parallel to the longitudinal axis of the shoe. In some aspects, the first line may extend from the end of the shoe at the toe area to the end of the shoe at the heel area. In some aspects, the first line may be curved.

According to certain aspects, the compartment system may also comprise a fourth medial compartment being adjustable from an unexpanded shape to an expanded shape. The fourth medial compartment may have a foot engaging portion that is substantially bounded by the first line, the second line, and at least one of the medial edge at the toe area and the lateral edge at the toe area, when the fourth medial compartment is in the expanded shape. The compartment system may also comprise a fourth lateral compartment being adjustable from an unexpanded shape to an expanded shape. The fourth lateral compartment may have a foot engaging portion that is substantially bounded by the first line, the second line, and at least one of the lateral edge at the toe area and the medial edge at the toe area, when the fourth lateral compartment is in the expanded shape.

In some aspects, at least one of the first medial compartment, the first lateral compartment, the second medial compartment, the second lateral compartment, the third medial compartment, the third lateral compartment, the fourth medial compartment, and the fourth lateral compartment comprises a valve configured to allow at least one of a gas and a liquid into the respective compartment.

According to certain aspects, at least one of the first medial compartment, the first lateral compartment, the second medial compartment, the second lateral compartment, the third medial compartment, and the third lateral compartment comprises an inner wall and an outer wall. The inner wall and the outer wall may be substantially parallel to the longitudinal axis of the shoe. In some aspects, when the at least one of the first medial compartment, the first lateral compartment, the second medial compartment, the second lateral compartment, the third medial compartment, and the third lateral compartment is in the expanded shape, an average height of the outer wall is greater than an average height of the inner wall such that an angle of elevation from the inner wall at its average height to the outer wall at its average height is at least about 26 degrees.

In some aspects, the first medial compartment and the first lateral compartment each comprise an inner wall and an outer wall. The inner wall and the outer wall may be substantially parallel to the longitudinal axis of the shoe. In some aspects, when the first medial compartment and the first lateral compartment are in their respective expanded shapes, an average height of the outer wall of the first lateral compartment is greater than an average height of the outer wall of the first medial compartment such that an angle of elevation from the outer wall of the first medial compartment at its average height to the outer wall of the first lateral compartment at its average height is at least about 17 degrees.

In some aspects, the first medial compartment and the first lateral compartment each comprise an inner wall and an outer wall. The inner wall and the outer wall may be substantially parallel to the longitudinal axis of the shoe. In some aspects, when the first medial compartment and the first lateral compartment are in their respective expanded shapes, an average height of the outer wall of the first medial compartment is greater than an average height of the outer wall of the first lateral compartment such that an angle of elevation from the outer wall of the first lateral compartment at its average height to the outer wall of the first medial compartment at its average height is at least about 17 degrees.

In some aspects, the second medial compartment and the second lateral compartment each comprise an inner wall and an outer wall. The inner wall and the outer wall may be substantially parallel to the longitudinal axis of the shoe. In some aspects, when the second medial compartment and the second lateral compartment are in their respective expanded shapes, an average height of the outer wall of the second lateral compartment is greater than an average height of the outer wall of the second medial compartment such that an angle of elevation from the outer wall of the second medial compartment at its average height to the outer wall of the second lateral compartment at its average height is at least about 17 degrees.

In some aspects, the second medial compartment and the second lateral compartment each comprise an inner wall and an outer wall. The inner wall and the outer wall may be substantially parallel to the longitudinal axis of the foot. In some aspects, when the second medial compartment and the second lateral compartment are in their respective expanded shapes, an average height of the outer wall of the second medial compartment is greater than an average height of the outer wall of the second lateral compartment such that an angle of elevation from the outer wall of the second lateral compartment at its average height to the outer wall of the second medial compartment at its average height is at least about 17 degrees.

According to certain aspects, at least a portion of the compartment system is further configured to be inflatable with at least one of a gas and a liquid. In some aspects, as the compartment system is adjusted from the first shape to the second shape, at least one of the one or more elongate members becomes progressively taut along its body. In some aspects, the first state is not taut.

In some aspects, the first and second parts of the one of the one or more elongate members are parts of different elongate members. The one or more elongate members may comprise at least one of para-aramid synthetic fiber, nylon, polyethylene, polypropylene, polystyrene, polyvinylchloride, polymethylmethacrylate, and polymethylacrylate. At least a portion of the one or more elongate members may be elastic. The one or more elongate members may be disposed within an interior of the compartment system. The one or more elongate members may form a tether network having a cross-sectional pattern comprising at least one of a honeycomb pattern, a cobweb pattern, a mesh pattern, and a checkered pattern. In some aspects, when one of the elongate members is substantially taut, the elongate member counteracts at least one of a compressive force, an expansive force, and a lateral force, acting on the compartment system. In some aspects, when one of the elongate members is substantially taut, the elongate member reduces movement of a first portion of the compartment system relative to a second portion of the compartment system.

According to various aspects of the subject disclosure, a foot orthotic system for affecting a position of a foot is provided. The foot orthotic system may comprise a compartment system configured to engage a shoe and engage at least a portion of a foot when the foot resides in or on the shoe. The compartment system may be adjustable from a first shape to a second shape. The foot orthotic system may also comprise a tether network having a first section and a second section. The first section may be coupled to a first part of the compartment system. The second section may be coupled to a second part of the compartment system. In some aspects, as the compartment system is adjusted from the first shape to the second shape, the tether network changes from a first state to a second state that is more taut than is the first state. In some aspects, when the compartment system engages at least a portion of the foot and is adjusted from the first shape to the second shape, a position of the foot relative to the shoe is adjusted.

In some aspects, the tether network may comprise one or more elongate members. Each of the one or more elongate members may have a first portion, a second portion, and a body therebetween. The first portion may be coupled to at least one of the first part of the compartment system and a first part of one of the one or more elongate members. The second portion may be coupled to at least one of the second part of the compartment system and a second part of one of the one or more elongate members. In some aspects, as the compartment system is adjusted from the first shape to the second shape, at least one of the one or more elongate members changes from a first condition to a second condition that is more taut than is the first condition.

In some aspects, the tether network may also comprise a third section between the first section and the second section. In some aspects, as the compartment system is adjusted from the first shape to the second shape, the tether network may become progressively taut along the third section. In some aspects, the first state is not taut.

According to various aspects of the subject disclosure, a method for affecting a position of a foot is provided. The method may comprise may comprise contacting, with a foot, a compartment system that is engaged with a shoe. The compartment system may be adjustable from a first shape to a second shape. The method may also comprise adjusting the compartment system from the first shape to the second shape such that a tether network, having a first section coupled to a first part of the compartment system and a second section coupled to a second part of the compartment system, changes from a first state to a second state that is more taut than is the first state. In some aspects, adjusting the compartment system may result in a change of a position of the foot relative to the shoe.

In some aspects, the adjusting the compartment system comprises inflating the compartment system with at least one of a gas and a liquid. The method may also comprise counteracting, with the tether network, at least one of a compressive force, an expansive force, and a lateral force, acting on the compartment system. In some aspects, the method may also comprise reducing, with the tether network, movement of a first portion of the compartment system relative to a second portion of the compartment system.

According to various aspects of the subject disclosure, a foot orthotic system for affecting a position of a foot is provided. The foot orthotic system may comprise a compartment system configured to engage a shoe and engage at least a portion of a foot when the foot resides in or on the shoe. The compartment system may be adjustable from a first shape to a second shape. In some aspects, when the compartment system engages at least a portion of the foot and is adjusted from the first shape to the second shape, a position of the foot relative to the shoe is adjusted. In some aspects, the foot orthotic system may also comprise the shoe.

According to certain aspects, the compartment system may comprise a first line extending from at least one of (a) a medial edge of the shoe at a toe area of the shoe, and (b) a lateral edge of the shoe at the toe area, to at least one of (a) the medial edge at a heel area of the shoe, and (b) the lateral edge at the heel area. The medial edge may extend from an end of the shoe at the toe area to an end of the shoe at the heel area. The lateral edge may extend from the end of the shoe at the toe area to the end of the shoe at the heel area. The compartment system may also comprise a second line extending from the medial edge to the lateral edge. The second line may be substantially perpendicular to a longitudinal axis of the shoe. The longitudinal axis of the shoe may extend from the end of the shoe at the toe area to the end of the shoe at the heel area. The compartment system may also comprise a third line extending from the medial edge to the lateral edge. The third line may be substantially perpendicular to the longitudinal axis of the shoe and disposed between the second line and the heel area. The compartment system may also comprise a fourth line extending from the medial edge to the lateral edge. The fourth line may be substantially perpendicular to the longitudinal axis of the shoe and disposed between the third line and the heel area.

In some aspects, the compartment system may comprise a first medial compartment being adjustable from an unexpanded shape to an expanded shape. The first medial compartment may have a foot engaging portion that is substantially bounded by the first line, the second line, the third line, and the medial edge, when the first medial compartment is in the expanded shape. The compartment system may also comprise a first lateral compartment being adjustable from an unexpanded shape to an expanded shape. The first lateral compartment may have a foot engaging portion that is substantially bounded by the first line, the second line, the third line, and the lateral edge, when the first lateral compartment is in the expanded shape.

In some aspects, the compartment system may also comprise a second medial compartment being adjustable from an unexpanded shape to an expanded shape. The second medial compartment may have a foot engaging portion that is substantially bounded by the first line, the third line, the fourth line, and the medial edge, when the second medial compartment is in the expanded shape. The compartment system may also comprise a second lateral compartment being adjustable from an unexpanded shape to an expanded shape. The second lateral compartment may have a foot engaging portion that is substantially bounded by the first line, the third line, the fourth line, and the lateral edge, when the second lateral compartment is in the expanded shape.

In some aspects, the compartment system may also comprise a third medial compartment being adjustable from an unexpanded shape to an expanded shape. The third medial compartment may have a foot engaging portion that is substantially bounded by the first line, the fourth line, and at least one of the medial edge at the heel area and the lateral edge at the heel area, when the third medial compartment is in the expanded shape. The compartment system may also comprise a third lateral compartment being adjustable from an unexpanded shape to an expanded shape. The third lateral compartment may have a foot engaging portion that is substantially bounded by the first line, the fourth line, and at least one of the lateral edge at the heel area and the medial edge at the heel area, when the third lateral compartment is in the expanded shape.

According to certain aspects, at least one of the first medial compartment, the first lateral compartment, the second medial compartment, the second lateral compartment, the third medial compartment, and the third lateral compartment comprises an inner wall and an outer wall. The inner wall and the outer wall may be substantially parallel to the longitudinal axis of the shoe. In some aspects, when the at least one of the first medial compartment, the first lateral compartment, the second medial compartment, the second lateral compartment, the third medial compartment, and the third lateral compartment is in the expanded shape, an average height of the outer wall is greater than an average height of the inner wall such that an angle of elevation from the inner wall at its average height to the outer wall at its average height is at least about 26 degrees.

In certain aspects, as measured along a length of the shoe, a distance between the end of the shoe at the toe area and the second line, a distance between the second line and the third line, a distance between the third line and the fourth line, and a distance between the fourth line and the end of the shoe at the heel area are substantially equal. In some aspects, the second line may be spaced such that between about 20% and about 35% of a length of the shoe lies between the end of the shoe at the toe area and the second line. The third line may be spaced such that between about 45% and about 55% of a length of the shoe lies between the end of the shoe at the toe area and the third line. The fourth line may be spaced such that between about 70% and about 85% of a length of the shoe lies between the end of the shoe at the toe area and the fourth line.

In some aspects, the respective foot engaging portions of the first medial compartment, the first lateral compartment, the second medial compartment, the second lateral compartment, the third medial compartment, and the third lateral compartment are configured to engage at least 70% of an under side of the foot. In some aspects, the first line may be substantially parallel to the longitudinal axis of the shoe. In some aspects, the first line may extend from the end of the shoe at the toe area to the end of the shoe at the heel area. In some aspects, the first line may be curved.

According to certain aspects, the compartment system may also comprise a fourth medial compartment being adjustable from an unexpanded shape to an expanded shape. The fourth medial compartment may have a foot engaging portion that is substantially bounded by the first line, the second line, and at least one of the medial edge at the toe area and the lateral edge at the toe area, when the fourth medial compartment is in the expanded shape. The compartment system may also comprise a fourth lateral compartment being adjustable from an unexpanded shape to an expanded shape. The fourth lateral compartment may have a foot engaging portion that is substantially bounded by the first line, the second line, and at least one of the lateral edge at the toe area and the medial edge at the toe area, when the fourth lateral compartment is in the expanded shape.

In some aspects, at least one of the first medial compartment, the first lateral compartment, the second medial compartment, the second lateral compartment, the third medial compartment, the third lateral compartment, the fourth medial compartment, and the fourth lateral compartment comprises a valve configured to allow at least one of a gas and a liquid into the respective compartment.

In some aspects, the first medial compartment and the first lateral compartment each comprise an inner wall and an outer wall. The inner wall and the outer wall may be substantially parallel to the longitudinal axis of the shoe. In some aspects, when the first medial compartment and the first lateral compartment are in their respective expanded shapes, an average height of the outer wall of the first lateral compartment is greater than an average height of the outer wall of the first medial compartment such that an angle of elevation from the outer wall of the first medial compartment at its average height to the outer wall of the first lateral compartment at its average height is at least about 17 degrees.

In some aspects, the first medial compartment and the first lateral compartment each comprise an inner wall and an outer wall. The inner wall and the outer wall may be substantially parallel to the longitudinal axis of the shoe. In some aspects, when the first medial compartment and the first lateral compartment are in their respective expanded shapes, an average height of the outer wall of the first medial compartment is greater than an average height of the outer wall of the first lateral compartment such that an angle of elevation from the outer wall of the first lateral compartment at its average height to the outer wall of the first medial compartment at its average height is at least about 17 degrees.

In some aspects, the second medial compartment and the second lateral compartment each comprise an inner wall and an outer wall. The inner wall and the outer wall may be substantially parallel to the longitudinal axis of the shoe. In some aspects, when the second medial compartment and the second lateral compartment are in their respective expanded shapes, an average height of the outer wall of the second lateral compartment is greater than an average height of the outer wall of the second medial compartment such that an angle of elevation from the outer wall of the second medial compartment at its average height to the outer wall of the second lateral compartment at its average height is at least about 17 degrees.

In some aspects, the second medial compartment and the second lateral compartment each comprise an inner wall and an outer wall. The inner wall and the outer wall may be substantially parallel to the longitudinal axis of the foot. In some aspects, when the second medial compartment and the second lateral compartment are in their respective expanded shapes, an average height of the outer wall of the second medial compartment is greater than an average height of the outer wall of the second lateral compartment such that an angle of elevation from the outer wall of the second lateral compartment at its average height to the outer wall of the second medial compartment at its average height is at least about 17 degrees.

According to certain aspects, at least a portion of the compartment system is further configured to be inflatable with at least one of a gas and a liquid. The foot orthotic system may also comprise one or more elongate members. Each of the one or more elongate members may have a first portion, a second portion, and a body therebetween. The first portion may be coupled to at least one of a first part of the compartment system and a first part of one of the one or more elongate members. The second portion may be coupled to at least one of a second part of the compartment system and a second part of one of the one or more elongate members. In some aspects, as the compartment system is adjusted from the first shape to the second shape, at least one of the one or more elongate members changes from a first state to a second state that is more taut than is the first state.

In some aspects, as the compartment system is adjusted from the first shape to the second shape, at least one of the one or more elongate members becomes progressively taut along its body. In some aspects, the first state is not taut. In some aspects, the first and second parts of the one of the one or more elongate members are parts of different elongate members. The one or more elongate members may comprise at least one of para-aramid synthetic fiber, nylon, polyethylene, polypropylene, polystyrene, polyvinylchloride, polymethylmethacrylate, and polymethylacrylate. At least a portion of the one or more elongate members may be elastic. The one or more elongate members may be disposed within an interior of the compartment system. The one or more elongate members may form a tether network having a cross-sectional pattern comprising at least one of a honeycomb pattern, a cobweb pattern, a mesh pattern, and a checkered pattern. In some aspects, when one of the elongate members is substantially taut, the elongate member counteracts at least one of a compressive force, an expansive force, and a lateral force, acting on the compartment system. In some aspects, when one of the elongate members is substantially taut, the elongate member reduces movement of a first portion of the compartment system relative to a second portion of the compartment system.

Additional features and advantages of the invention will be set forth in the description below, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute a part of this specification, illustrate aspects of the invention and together with the description serve to explain the principles of the invention.

FIGS. 1A and 1B illustrate aerial views of examples of a compartment system, in accordance with various aspects of the subject disclosure.

FIGS. 2A and 2B illustrate examples of a compartment, in accordance with various aspects of the subject disclosure.

FIG. 3 illustrates an example of an elongate member, in accordance with various aspects of the subject disclosure.

FIGS. 4A and 4B illustrate an example of a compartment, in accordance with various aspects of the subject disclosure.

FIG. 5 illustrates an example of an elongate member being coupled to more than one compartment, in accordance with various aspects of the subject disclosure.

FIGS. 6A, 6B, 6C, 6D, and 6E illustrate various configurations of an elongate member, in accordance with various aspects of the subject disclosure.

FIG. 7 illustrates an example of a compartment system utilizing a tether network of one or more elongate members, in accordance with various aspects of the subject disclosure.

FIGS. 8A, 8B, 8C, 8D, 8E, 8F, 8G, 8H, 8I, 8J, and 8K illustrate examples of a compartment, in accordance with various aspects of the subject disclosure.

FIG. 9 illustrates an example of a method for affecting a position of a foot, in accordance with various aspects of the subject disclosure.

### DETAILED DESCRIPTION

In the following detailed description, numerous specific details are set forth to provide a full understanding of the present invention. It will be apparent, however, to one ordinarily skilled in the art that the present invention may be practiced without some of these specific details. In other instances, well-known structures and techniques have not been shown in detail so as not to obscure the present invention.

According to various aspects of the disclosure, systems and methods are provided to affect a position of a foot, for example, to counter excessive pronation and/or excessive supination of the foot. A foot orthotic system may be provided that comprises a compartment system configured to engage a shoe and engage at least a portion of a foot when the foot resides in or on the shoe. In some aspects, the compartment system may be coupled to the shoe or be integral with the shoe. For example, the compartment system may be a shoe insert or other suitable market shoe accessory. The compartment system may also be included in the assembly of a shoe insole. A shoe may include, for example and without limitation, boots, sneakers, sandals, flip-flips, or other suitable footwear. In some aspects, the foot orthotic system may comprise the shoe.

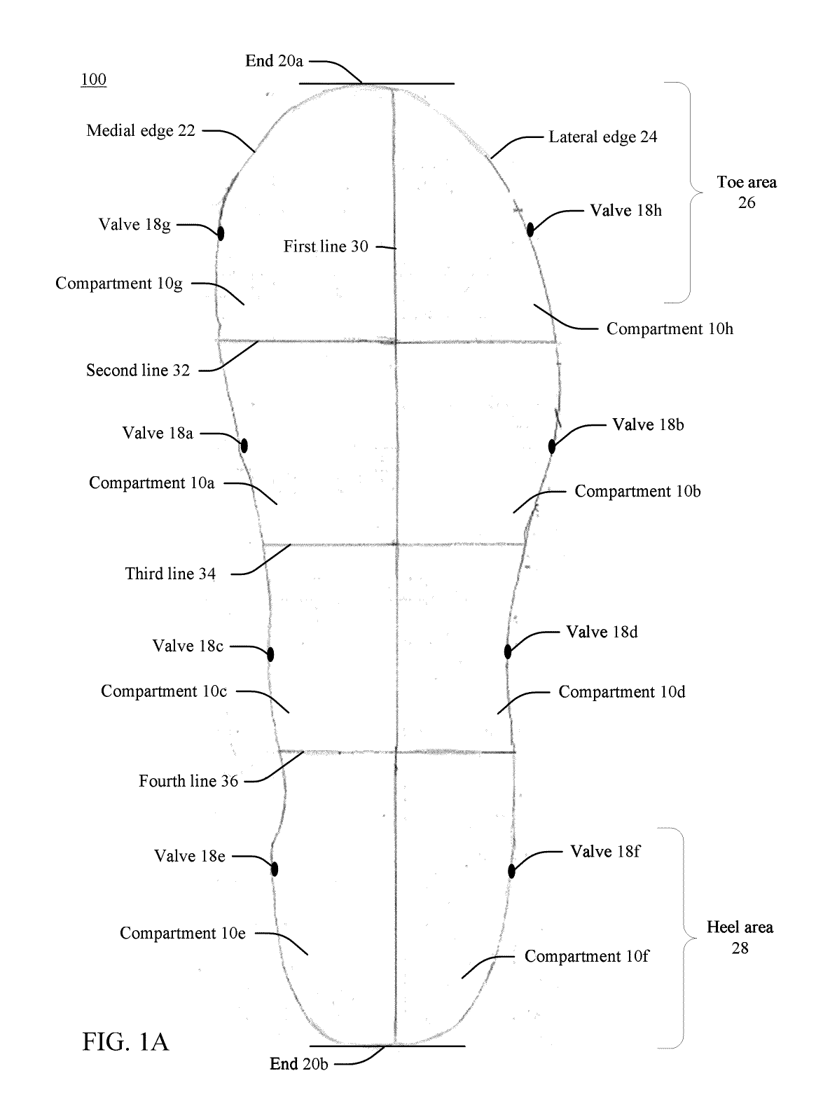

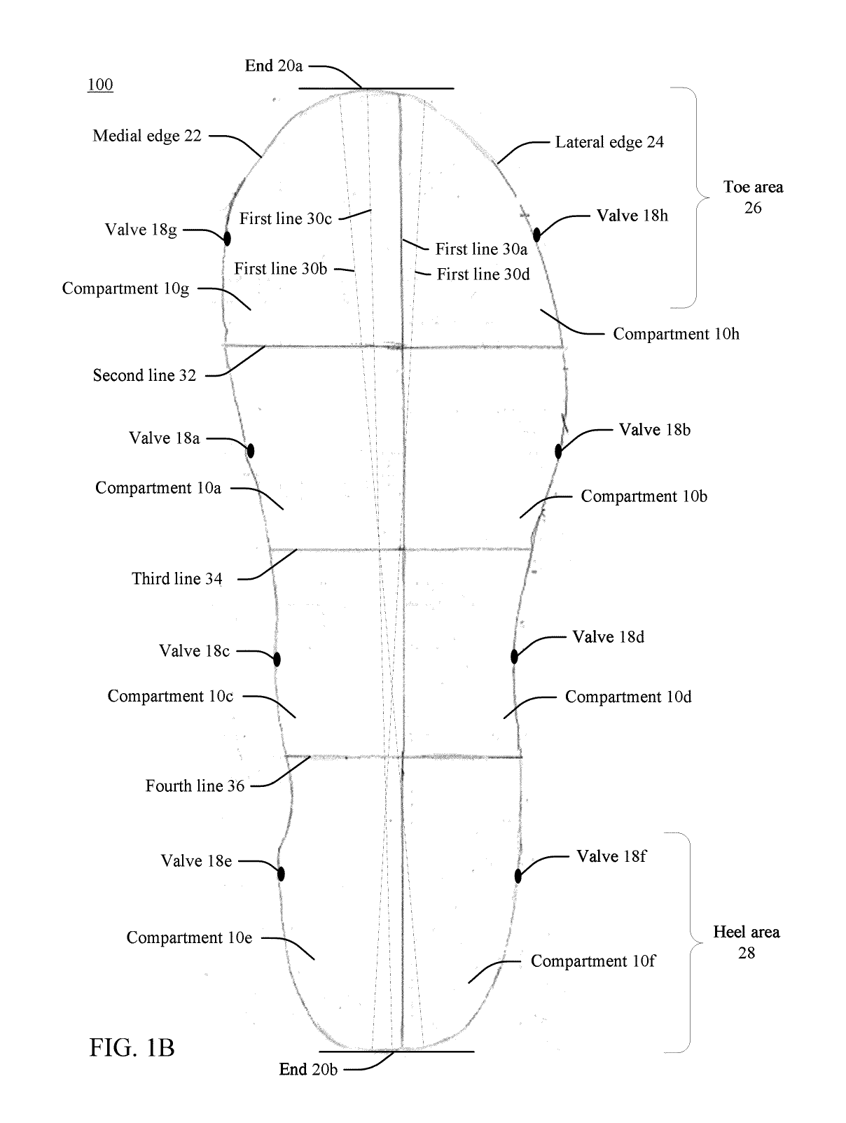

FIGS. 1A and 1B illustrate aerial views of examples of compartment system 100, in accordance with various aspects of the subject disclosure. Compartment system 100 may generally conform to an outline of a shoe. Although the outline of the shoe as shown is for a right foot, compartment system 100 may be equally applicable to a left foot using a mirror image of FIGS. 1A and 1B. In some aspects, compartment system 100 may be adjustable from a first shape to a second shape. Compartment system 100 may comprise one or more compartments 10 (e.g., compartments 10a, 10b, 10c, 10d, 10e, 10f, 10g, and 10h) that are each adjustable from a first shape to a second shape. At least a portion of compartment system 100 may be configured to be inflatable with at least one of a gas and a liquid. For example, each compartment 10 may comprise a valve 18 (e.g., valves 18a, 18b, 18c, 18d, 18e, 18f, 18g, and 18h) configured to allow at least one of the gas and the liquid into the compartment 10. When compartment system 100 engages at least a portion of the foot and is adjusted from the first shape to the second shape, a position of the foot relative to the shoe may be adjusted.

In some aspects, compartments 10 of compartment system 100 may be bounded to certain locations relative to a shoe. As shown in FIGS. 1A and 1B, a shoe may have an end 20a at a toe area 26 and an end 20b at a heel area 28. The shoe may include a medial edge 22 extending from end 20a to end 20b and a lateral edge 24 extending from end 20a to end 20b. Referring to FIG. 1A, compartment system 100 may comprise first line 30 extending from at least one of (a) medial edge 22 at toe area 26 and (b) lateral edge 24 at toe area 26, to at least one of (a) medial edge 22 at heel area 28 and (b) lateral edge 24 at heel area 28. Compartment system 100 may also comprise second line 32 extending from medial edge 22 to lateral edge 24. Second line 32 may be substantially perpendicular to a longitudinal axis of the shoe. In some aspects, the longitudinal axis of the shoe extends from end 20a to end 20b. Compartment system 100 may also comprise third line 34 extending from medial edge 22 to lateral edge 24. Third line 34 may be substantially perpendicular to the longitudinal axis of the shoe and disposed between second line 32 and heel area 28. Compartment system 100 may also comprise fourth line 36 extending from medial edge 22 to lateral edge 24. Fourth line 36 may be substantially perpendicular to the longitudinal axis of the shoe and disposed between third line 34 and heel area 28.

According to certain aspects, compartment system 100 may comprise a first medial compartment (e.g., compartment 10a) being adjustable from an unexpanded shape to an expanded shape. The first medial compartment may have a foot engaging portion that is substantially bounded by first line 30, second line 32, third line 34, and medial edge 22 when the first medial compartment is in the expanded shape. Compartment system 100 may also comprise a first lateral compartment (e.g., compartment 10b) being adjustable from an unexpanded shape to an expanded shape. The first lateral compartment may have a foot engaging portion that is substantially bounded by first line 30, second line 32, third line 34, and lateral edge 24 when the first lateral compartment is in the expanded shape. Compartment system 100 may also comprise a second medial compartment (e.g., compartment 10c) being adjustable from an unexpanded shape to an expanded shape. The second medial compartment may have a foot engaging portion that is substantially bounded by first line 30, third line 34, fourth line 36, and medial edge 22 when the second medial compartment is in the expanded shape. Compartment system 100 may also comprise a second lateral compartment (e.g., compartment 10d) being adjustable from an unexpanded shape to an expanded shape. The second lateral compartment may have a foot engaging portion that is substantially bounded by first line 30, third line 34, fourth line 36, and lateral edge 24 when the second lateral compartment is in the expanded shape.

According to certain aspects of the subject disclosure, compartment system 100 may also comprise a third medial compartment (e.g., compartment 10e) being adjustable from an unexpanded shape to an expanded shape. The third medial compartment may have a foot engaging portion that is substantially bounded by first line 30, fourth line 36, and at least one of medial edge 22 at heel area 28 and lateral edge 24 at heel area 28 when the third medial compartment is in the expanded shape. Compartment system 100 may also comprise a third lateral compartment (e.g., compartment 100 being adjustable from an unexpanded shape to an expanded shape. The third lateral compartment may have a foot engaging portion that is substantially bounded by first line 30, fourth line 36, and at least one of lateral edge 24 at heel area 28 and medial edge 22 at heel area 28 when the third lateral compartment is in the expanded shape.

In some aspects, compartment system 100 may also comprise a fourth medial compartment (e.g., compartment 10g) being adjustable from an unexpanded shape to an expanded shape. The fourth medial compartment may have a foot engaging portion that is substantially bounded by first line 30, second line 32, and at least one of medial edge 22 at toe area 26 and lateral edge 24 at toe area 26 when the fourth medial compartment is in the expanded shape. Compartment system 100 may also comprise a fourth lateral compartment (e.g., compartment 10h) being adjustable from an unexpanded shape to an expanded shape. The fourth lateral compartment may have a foot engaging portion that is substantially bounded by first line 30, second line 32, and at least one of lateral edge 24 at toe area 26 and medial edge 22 at toe area 26 when the fourth lateral compartment is in the expanded shape.

According to various aspects of the subject disclosure, first line 30, second line 32, third line 34, and fourth line 36 may be disposed in various positions in order to form the boundaries of compartments 10. For example, referring to FIG. 1B, first line 30 (e.g., first line 30a, 30b, 30c, and 30d) may be oriented at different angles as shown. In some aspects, first line 30 may be substantially parallel to the longitudinal axis of the shoe. In some aspects, first line 30 may extend from end 20a to end 20b. In some aspects, at least one of first line 30, second line 32, third line 34, and fourth line 36 may be curved.

In certain aspects, as measured along a length of the shoe, a distance between end 20a and second line 32, a distance between second line 32 and third line 34, a distance between third line 34 and fourth line 36, and a distance between fourth line 36 and end 20b are substantially equal. In some aspects, second line 32 may be spaced such that between about 20% and about 35% of a length of the shoe lies between end 20a and second line 32. In some aspects, second line 32 may be spaced such that less than about 20% or greater than about 35% of a length of the shoe lies between end 20a and second line 32. In some aspects, third line 34 may be spaced such that between about 45% and about 55% of a length of the shoe lies between end 20a and third line 34. In some aspects, third line 34 may be spaced such that less than about 45% or greater than about 55% of a length of the shoe lies between end 20a and third line 34. In some aspects, fourth line 36 is spaced such that between about 70% and about 85% of a length of the shoe lies between end 20a and fourth line 36. In some aspects, fourth line 36 is spaced such that less than about 70% or greater than about 85% of a length of the shoe lies between end 20a and fourth line 36.

According to various aspects of the subject disclosure, the respective foot engaging portions of compartment 10a, compartment 10b, compartment 10c, compartment 10d, compartment 10e, and compartment 10f may be configured to engage at least 70% of an under side of the foot. In some aspects, the respective foot engaging portions of compartment 10a, compartment 10b, compartment 10c, compartment 10d, compartment 10e, and compartment 10f may be configured to engage at least 60% of an under side of the foot.

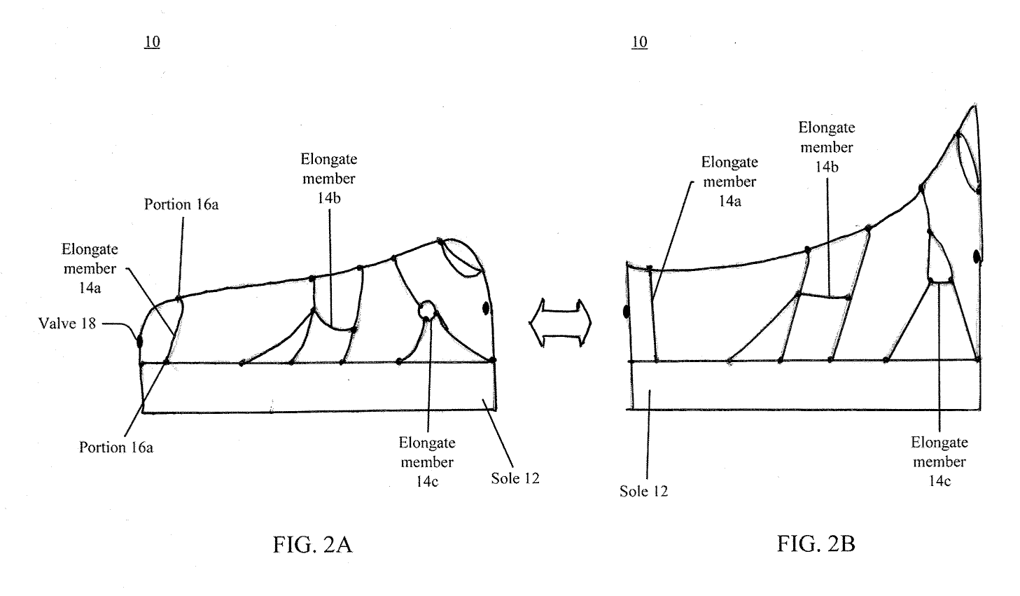

FIGS. 2A and 2B illustrate examples of compartment 10, in accordance with various aspects of the subject disclosure. FIGS. 2A and 2B provide side views of compartment 10 as viewed from either end 20a or end 20b. In some aspects, compartment 10 may comprise sole 12. In some aspects, compartment 10 may be separate from a shoe. Compartment 10 may be adjusted from a first shape (e.g., as shown in FIG. 2A) to a second shape (e.g., as shown in FIG. 2B), and vice versa. In some aspects, the foot orthotic system may also comprise one or more elongate members 14 (e.g., elongate members 14a, 14b, 14c, 14d, 14e). An elongate member 14 may be coupled to various parts of compartment 10 and/or to other elongate members 14 in order to increase the stability of compartment 10. For example, when elongate member 14 is substantially taut, elongate member 14 may counteract at least one of a compressive force, an expansive force, and a lateral force, acting on compartment 10. In one example, when elongate member 14 is substantially taut, elongate member 14 may reduce movement of a first portion of compartment 10 relative to a second portion of compartment 10. In some aspects, elongate members 14 may be disposed within an interior of compartment 10 and/or external to compartment 10.

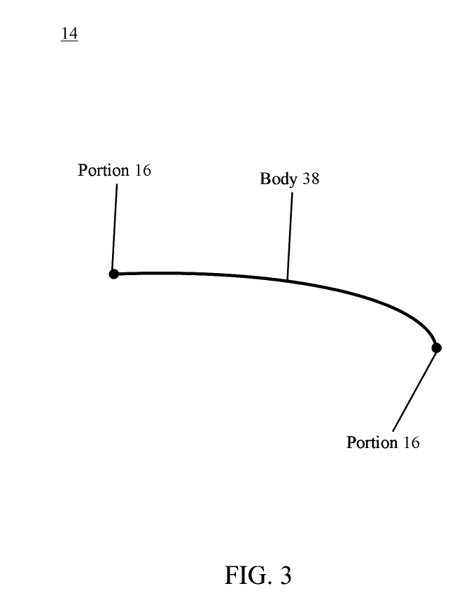

FIG. 3 illustrates an example of an elongate member 14, in accordance with various aspects of the subject disclosure. Elongate member 14 may comprise a first portion 16, a second portion 16, and a body 38 therebetween. At least a portion of elongate member 14 may be elastic. In some aspects, elongate member 14 may be at least one of a string, a cord, a ribbon, a rope, a wire, a cable, a thread, and a chain. In some aspects, elongate member 14 may comprise at least one of para-aramid synthetic fiber (e.g., Kevlar), nylon, polyethylene, polypropylene, polystyrene, polyvinylchloride, polymethylmethacrylate, polymethylacrylate, and other suitable materials known to those of skill in the art.

Referring back to FIGS. 2A and 2B, each elongate member 14 may have a first portion (e.g., portion 16a) coupled to at least one of a first part of compartment 10 and a first part of one of the one or more elongate members 14. Each elongate member 14 may have a second portion coupled to at least one of a second part of compartment 10 and a second part of one of the one or more elongate members 14. In some aspects, the first and second parts of the one of the one or more elongate members are parts of different elongate members. In one example, elongate member 14a has a portion 16a coupled to a part of compartment 10 and another portion 16a coupled to another part of compartment 10. In another example, elongate member 14b has a portion coupled to one elongate member and another portion coupled to another elongate member. In another example, elongate member 14c has a portion coupled to itself, thus forming at least a ring. Thus, in some aspects, other elongate members may be coupled to a body of elongate member 14c.

According to various aspects of the subject disclosure, as compartment 10 is adjusted from the first shape (e.g., as shown in FIG. 2A) to the second shape (e.g., as shown in FIG. 2B), at least one of the elongate members 14 changes from a first state to a second state that is more taut than is the first state. For example, elongate member 14a may have some slack as shown in FIG. 2A. As compartment 10 is adjusted to the shape as shown in FIG. 2B, elongate member 14a may become more taut. In some aspects, the first state is not taut. In some aspects, as compartment 10 is adjusted from the first shape to the second shape, eat least one of the elongate members 14 becomes progressively taut along its body.

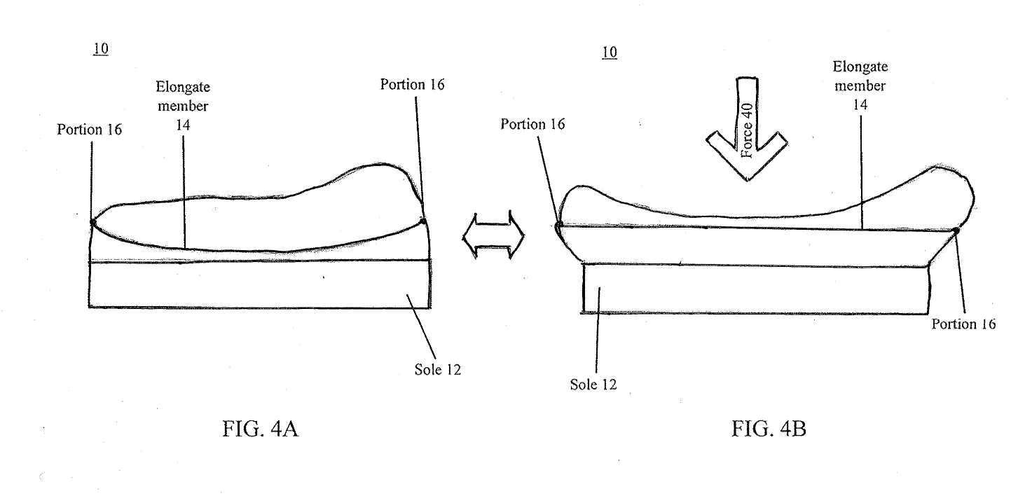

FIGS. 4A and 4B illustrate an example of compartment 10, in accordance with various aspects of the subject disclosure. Portions 16 of elongate member 14 are coupled to respective parts of compartment 10. Elongate member 14 is shown as having some slack in FIG. 4A, where compartment 10 is in a first shape. In FIG. 4B, compartment 10 is shown as being adjusted to a second shape. For example, a force 40 (e.g., a compressive force in a vertical direction) may be exerted on a top part of compartment 10 (e.g., a foot engaging portion of compartment 10), thus causing an expansive force in the horizontal direction and adjusting the shape of compartment 10 into a second shape. As compartment 10 is adjusted, elongate member 14 changes from a first state to a second state that is more taut than is the first state. As shown in FIG. 4B, elongate member 14 may counteract the expansive force in the horizontal direction as well as force 40 in the vertical direction. In some aspects, elongate member 14 may also counteract a lateral force acting on compartment 10.

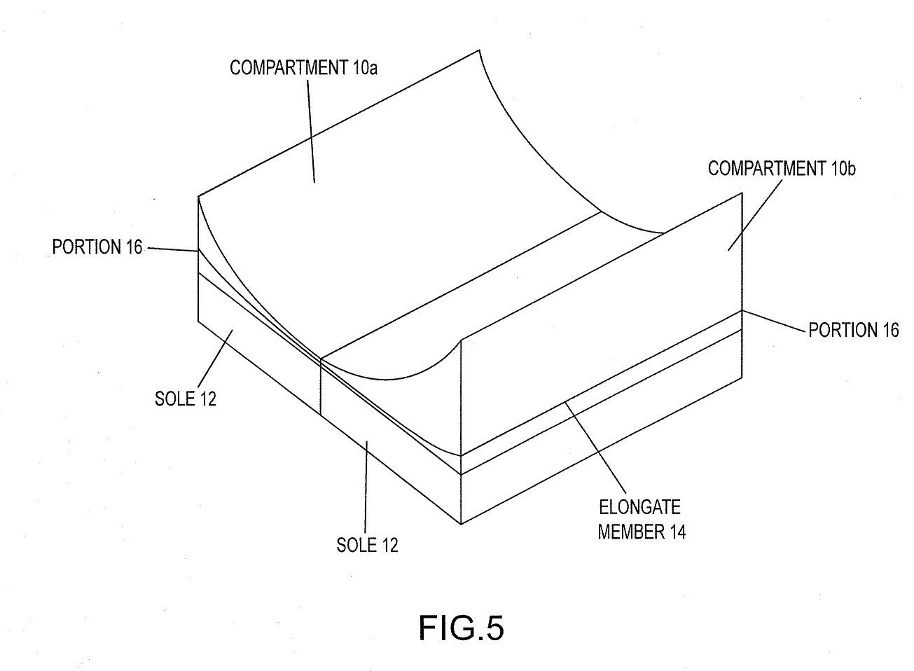

FIG. 5 illustrates an example of elongate member 14 being coupled to more than one compartment 10 (e.g., compartments 10a and 10b), in accordance with various aspects of the subject disclosure. Elongate member 14 may have a portion 16 coupled to one compartment 10a, and another portion 16 coupled to another compartment 10b. In some aspects, elongate member 14 may be external to compartments 10.

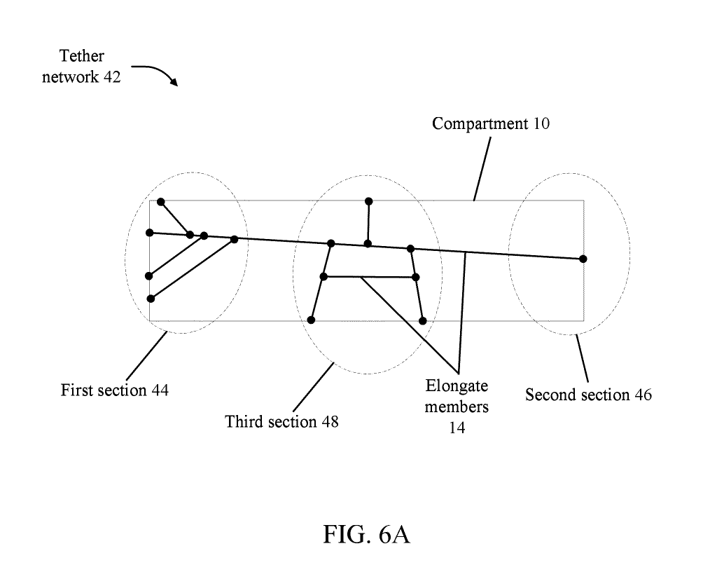

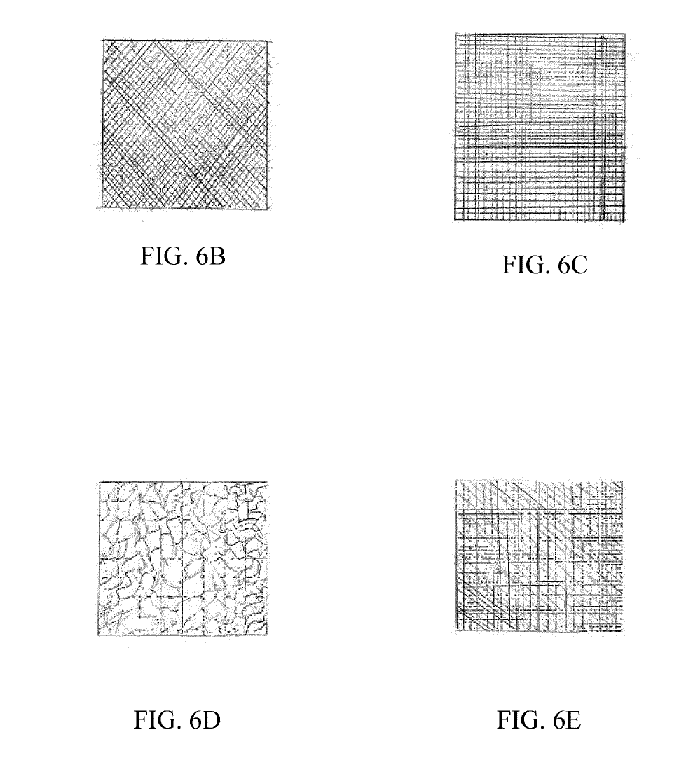

FIGS. 6A, 6B, 6C, 6D, and 6E illustrate various configurations of elongate member 14, in accordance with various aspects of the subject disclosure. In some aspects, one or more elongate members 14 may form a tether network 42. For example, as shown in FIG. 6A, a tether network 42 of one or more elongate members 14 may be coupled to compartment 10. Tether network 42 may comprise first section 44, second section 46, and third section 48 therebetween. Each of the sections may comprise one or more elongate members 14 or a portion of an elongate member 14. For example, first section 44, as shown in FIG. 6A, comprises one or more elongate members 14 having portions coupled to a part of compartment 10. Second section 46 comprises a portion of elongate member 14 coupled to another part of compartment 10. Third section 48 comprises one or more elongate members 14 disposed between first section 44 and second section 46. In some aspects, as compartment 10 is adjusted from a first shape to a second shape, tether network 42 changes from a first state to a second state that is more taut than is the first state. In some aspects, as compartment 10 is adjusted from the first shape to the second shape, tether network 42 becomes progressively taut along the third section. In some aspects, the first state is not taut.

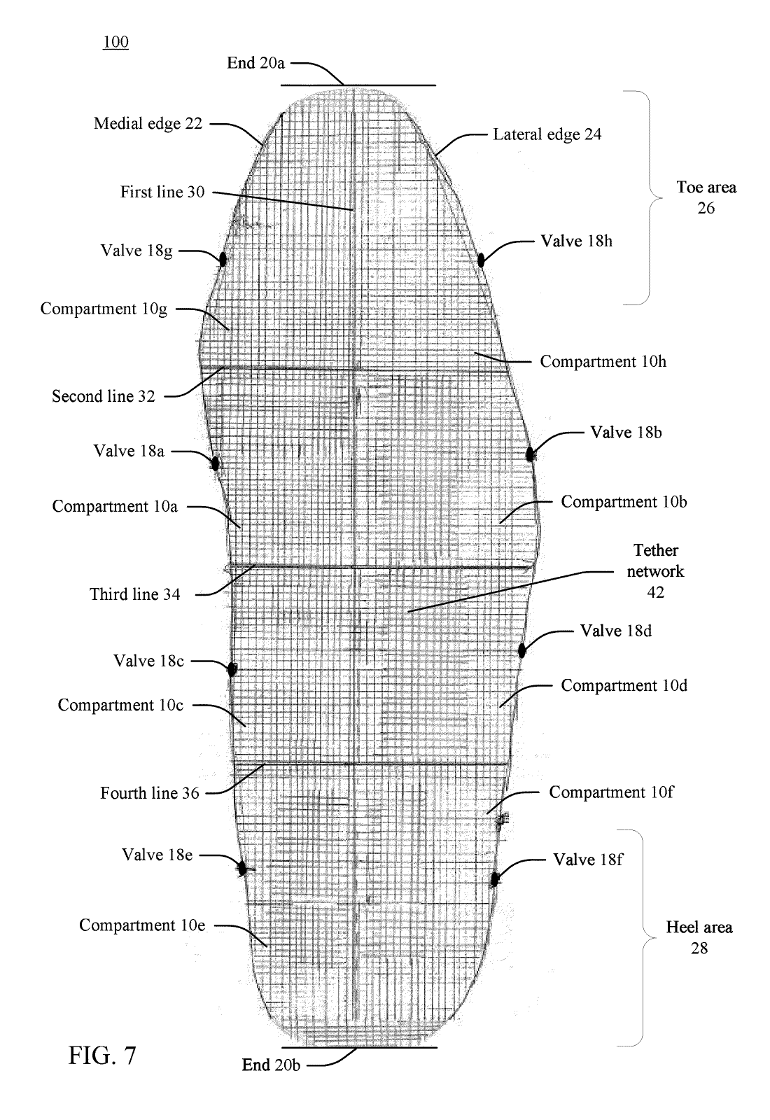

FIGS. 6B, 6C, 6D, and 6E illustrate various cross-sectional patterns of tether network 42, in accordance with various aspects of the subject disclosure. For example, tether network 42 may have a cross-sectional pattern comprising at least one of a honeycomb pattern, a cobweb pattern, a mesh pattern, and a checkered pattern. FIG. 7 illustrates an example of compartment system 100 utilizing a tether network 42 of one or more elongate members, in accordance with various aspects of the subject disclosure.

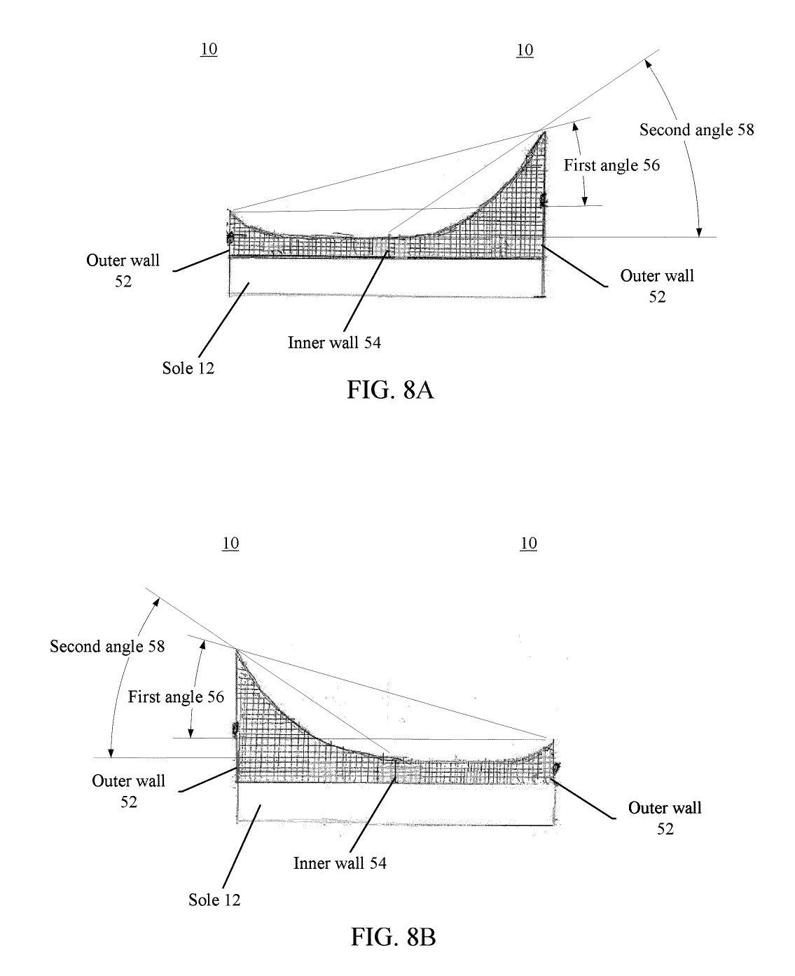

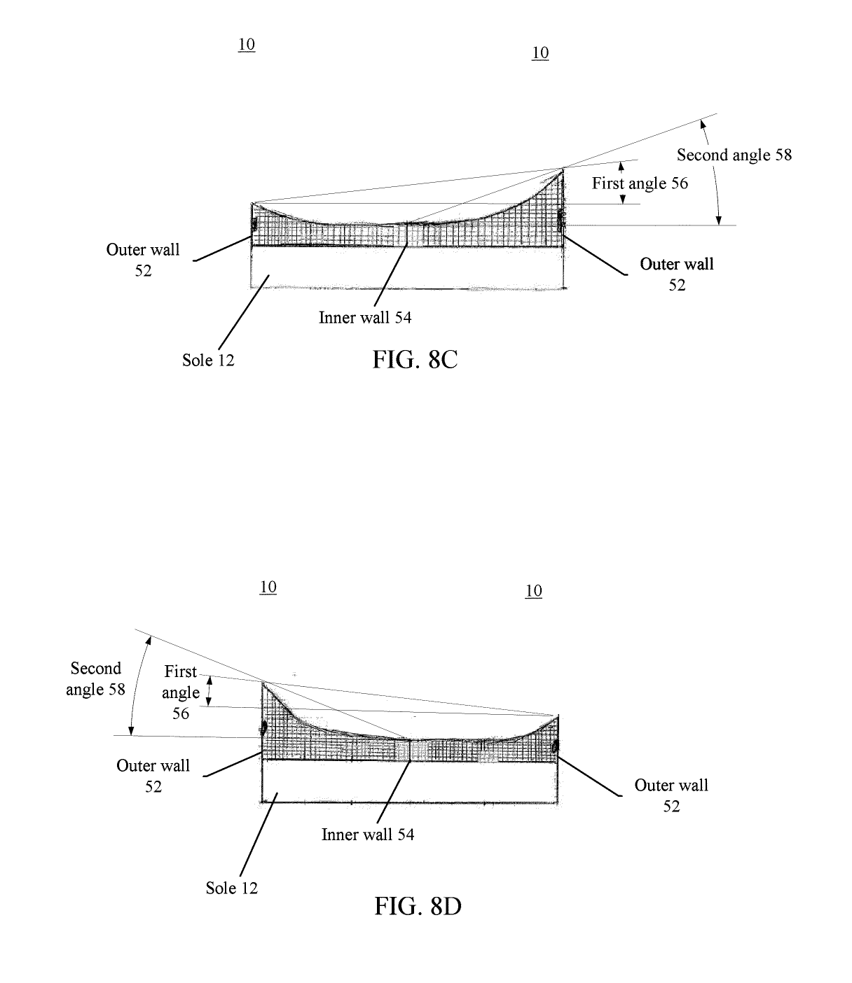

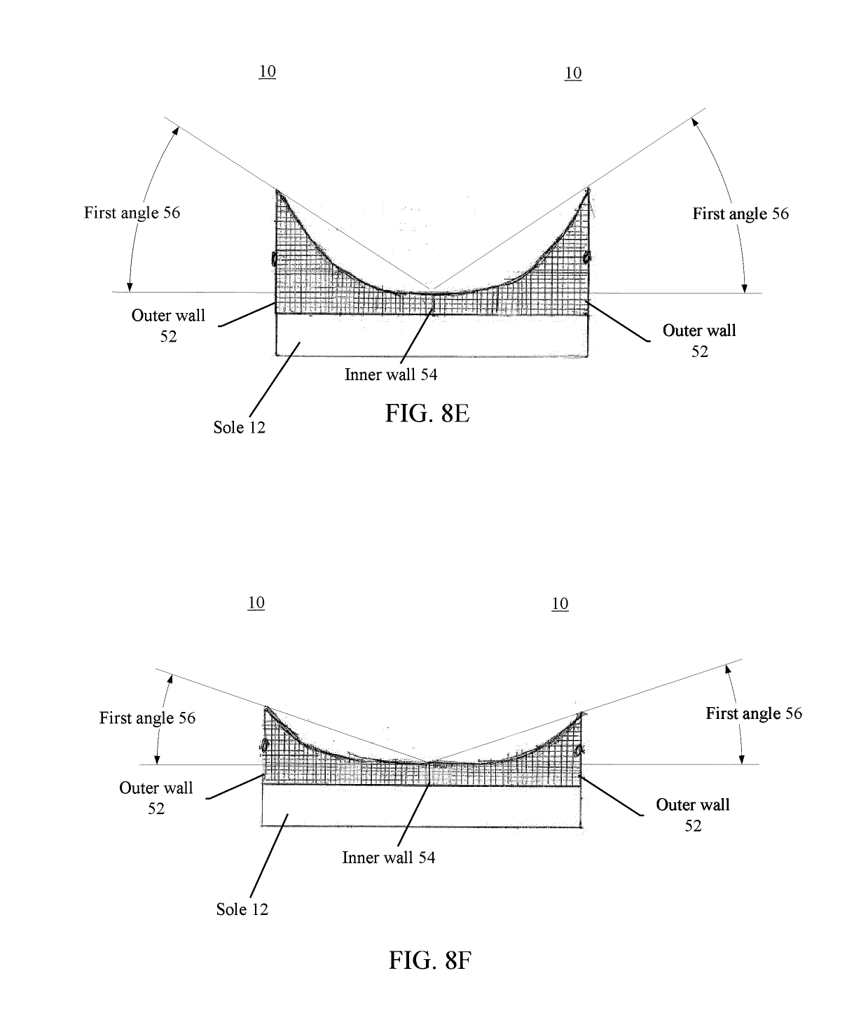

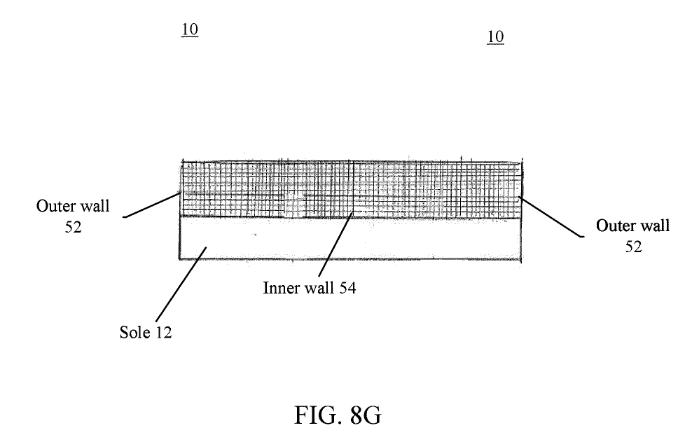

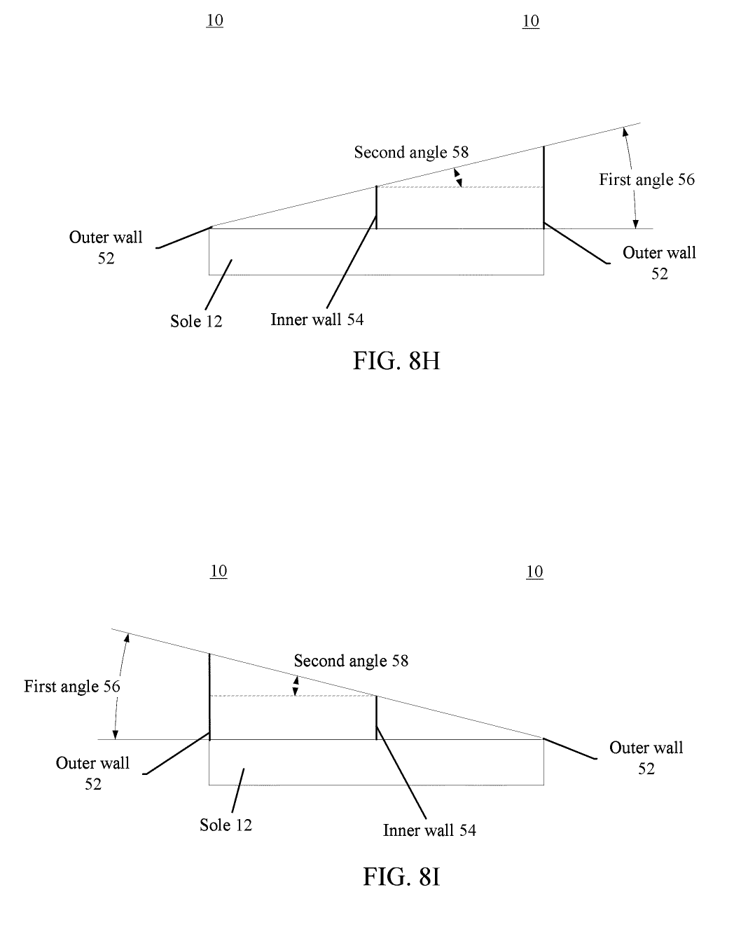

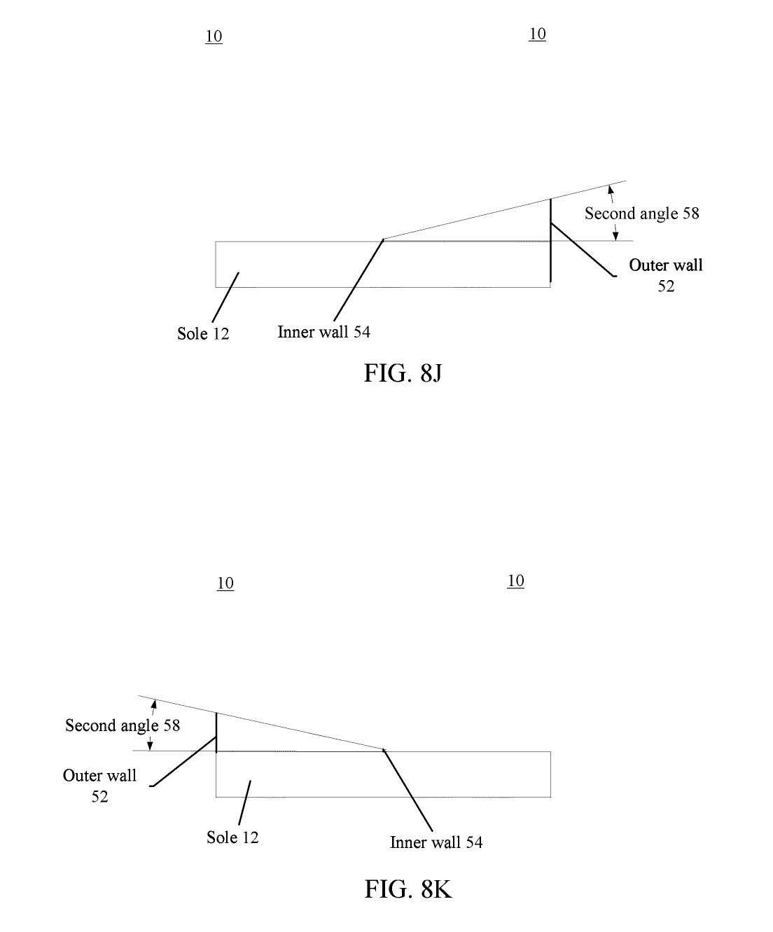

FIGS. 8A, 8B, 8C, 8D, 8E, 8F, 8G, 8H, 8I, 8J, and 8K illustrate examples of compartment 10 (e.g., compartments 10a, 10b, 10c, 10d, 10e, 10f, 10g, and 10h), in accordance with various aspects of the subject disclosure. FIGS. 8A, 8B, 8C, 8D, 8E, 8F, 8G, 8H, and 81 provide side views of a pair of compartments 10 (e.g., compartments 10a and 10b; 10c and 10d; 10e and 10f; or 10g and 10h) as viewed from end 20b. Each compartment 10 may comprise an outer wall 52 and an inner wall 54. In some aspects, outer wall 52 and inner wall 54 may be substantially parallel to the longitudinal axis of the shoe. Each compartment 10 of compartment system 100 may be adjusted to various shapes. In some aspects, the shape of each compartment 10 may be adjusted such that an angle of elevation from outer wall 52 of one compartment 10 at its average height to outer wall 52 of an opposite compartment 10 at its average height may be correspondingly adjusted. This angle may be referred to as first angle 56. In some aspects, the shape of each compartment 10 may be adjusted such that an angle of elevation from inner wall 54 at its average height to outer wall 52 at its average height may be correspondingly adjusted. This angle may be referred to as second angle 58.

FIGS. 8A, 8B, 8C, 8D, 8H, and 81 illustrate various shapes that each compartment 10 may be adjusted to. In some aspects, when a first compartment 10 (e.g., compartment 10 shown on either the right side or the left side of FIGS. 8A, 8B, 8C, or 8D) and a second compartment 10 (e.g., the compartment shown on the opposite side of first compartment 10) are in their respective expanded shapes, an average height of outer wall 52 of the first compartment 10 is greater than an average height of outer wall 52 of the second compartment 10 such that first angle 56 may be at least about 17 degrees. In some aspects, when the first compartment 10 and the second compartment 10 are in their respective expanded shapes, an average height of outer wall 52 of the first compartment 10 is greater than an average height of outer wall 52 of the second compartment 10 such that first angle 56 may be at least about 21 degrees. In some aspects, when the first compartment 10 and the second compartment 10 are in their respective expanded shapes, an average height of outer wall 52 of the first compartment 10 is greater than an average height of outer wall 52 of the second compartment 10 such that first angle 56 may be at least about 24 degrees. In some aspects, these angles of elevation may advantageously allow for the treatment of flat feet associated with pronation or “over arching” associated with supination.

In some aspects, when compartment 10 is in an expanded shape, an average height of its outer wall 52 may be greater than an average height of its inner wall 54 such that second angle 58 may be at least about 26 degrees. In some aspects, when compartment 10 is in an expanded shape, an average height of its outer wall 52 may be greater than an average height of its inner wall 54 such that second angle 58 may be at least about 30 degrees. In some aspects, when compartment 10 is in an expanded shape, an average height of its outer wall 52 may be greater than an average height of its inner wall 54 such that second angle 58 may be at least about 34 degrees. In some aspects, these angles of elevation may advantageously allow for the treatment of flat feet associated with pronation or “over arching” associated with supination.

FIGS. 8H and 8I illustrate examples of compartments 10 when second angle 58 is the same angle as first angle 56. In some aspects, second angle 58 may also be at least about 17 degrees. FIGS. 8J and 8K illustrate examples of using only one compartment with an expanded shape on either the medial side or the lateral side of a shoe, while the opposite side of the shoe may have a compartment 10 in an unexpanded shape or not have a compartment at all. In some aspects, second angle 58 may be at least about 17 degrees. In some aspects, second angle 58 may be at least about 21 degrees. In some aspects, second angle 58 may be at least about 24 degrees. In some aspects, second angle 58 may be at least about 26 degrees. In some aspects, second angle 58 may be at least about 30 degrees. In some aspects, second angle 58 may be at least about 34 degrees. In some aspects, these angles of elevation may advantageously allow for the treatment of flat feet associated with pronation or “over arching” associated with supination.

According to certain aspects, the shape of each compartment 10 may be adjusted to affect a position of the foot, for example, to counter excessive pronation and/or excessive supination of the foot. For example, the configuration of the compartments 10 shown in FIGS. 8A, 8C, 8H, and 8J may be utilized to counter supination of a right foot. The configuration of the compartments 10 shown in FIGS. 8B, 8D, 81, and 8K may be utilized to counter pronation of a right foot. The configuration of the compartments 10 shown in FIGS. 8E and 8F may be utilized to counter pronation of a foot (e.g., when the medial compartment is expanded) or supination of a foot (e.g., when the lateral compartment is expanded).

In some aspects, the pair of compartments 10a and 10b, in addition to the pair of compartments 10c and 10d, may utilize the configurations shown in FIGS. 8A, 8B, 8C, 8D, 8E, 8F, 8H, 8I, 8J, and 8K. In some aspects, the pair of compartments 10e and 10f may utilize the configurations shown in FIGS. 8C, 8D, 8F, 8H, 8I, 8J, and 8K. In some aspects, each compartment 10 may utilize any of the configurations of the compartments 10 shown in FIGS. 8A, 8B, 8C, 8D, 8E, 8F, 8G, 8H, 8I, 8J, and 8K. In some aspects, compartment system 100 may comprise any combination of compartments 10 utilizing any of the configurations shown in FIGS. 8A, 8B, 8C, 8D, 8E, 8F, 8G, 8H, 8I, 8J, and 8K. For example, compartment system 100 may comprise one or more compartments 10 on the medial side only (e.g., compartments 10a, 10c, 10e, and/or 10g). Compartment system 100 may also comprise one or more compartments 10 on the lateral side only (e.g., compartments 10b, 10d, 10f, and/or 10h).

Adjusting a shape of a compartment 10 and affecting a position of a foot may result in the foot being off balance. For example, if compartments 10c and 10d were adjusted to a shape to elevate respective portions of a foot, the foot may tilt forward, thus putting more weight on the toe area of the foot, which may possibly cause calluses in that area. The configuration of the compartments 10 shown in FIG. 8G may be utilized to reduce any off balances of the foot resulting from adjusting other compartments 10. In such a configuration, each compartment 10 may be adjusted such that the compartment 10 is elevated uniformly. Thus, such a configuration may be used in a compartment 10 where a portion of the foot that the compartment 10 is engaged to is off balance (e.g., with excess weight exerted on it). In this way, the compartment 10 may be elevated to alleviate the off balance. In some aspects, the pair of compartments 10e and 10f, in addition to the pair of compartments 10g and 10h, may utilize the configuration shown in FIG. 8G.

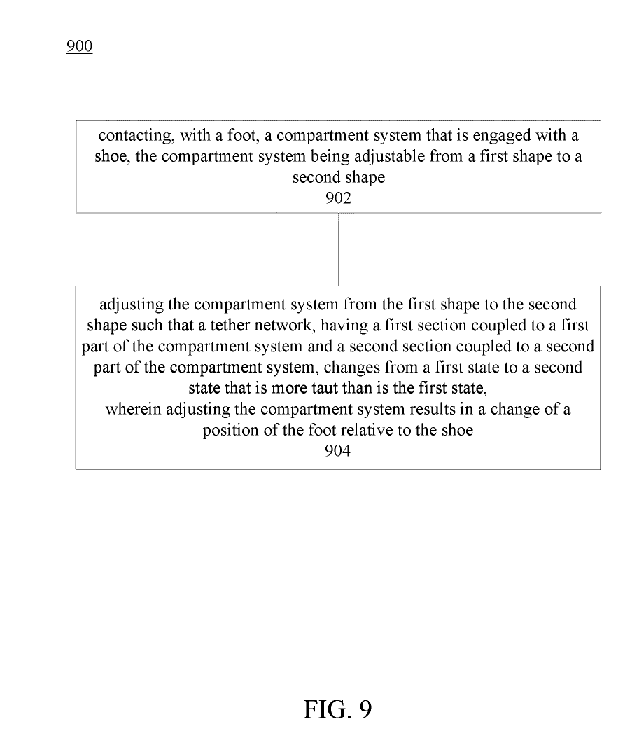

FIG. 9 illustrates an example of a method 900 for affecting a position of a foot, in accordance with various aspects of the subject disclosure. Method 900 may comprise contacting, with a foot, a compartment system that is engaged with a shoe (902). The compartment system may be adjustable from a first shape to a second shape. Method 900 may also comprise adjusting the compartment system from the first shape to the second shape such that a tether network, having a first section coupled to a first part of the compartment system and a second section coupled to a second part of the compartment system, changes from a first state to a second state that is more taut than is the first state (904). Adjusting the compartment system may result in a change of a position of the foot relative to the shoe.

The foregoing description is provided to enable a person skilled in the art to practice the various configurations described herein. While the present invention has been particularly described with reference to the various figures and configurations, it should be understood that these are for illustration purposes only and should not be taken as limiting the scope of the invention.

There may be many other ways to implement the invention. Various functions and elements described herein may be partitioned differently from those shown without departing from the scope of the invention. Various modifications to these configurations will be readily apparent to those skilled in the art, and generic principles defined herein may be applied to other configurations. Thus, many changes and modifications may be made to the invention, by one having ordinary skill in the art, without departing from the scope of the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

What is claimed is:

1. A foot orthotic system for affecting a position of a foot, the foot orthotic system comprising:
a compartment system configured to engage a shoe and engage at least a portion of a foot when the foot resides in or on the shoe, the compartment system being adjustable from a first shape to a second shape; and
one or more elongate members, each of the one or more elongate members having a first portion, a second portion, and a body therebetween, the first portion coupled to at least one of a first part of the compartment system and a first part of one of the one or more elongate members, the second portion coupled to at least one of a second part of the compartment system and a second part of one of the one or more elongate members,
wherein, as the compartment system is adjusted from the first shape to the second shape, at least one of the one or more elongate members changes from a first state to a second state that is more taut than is the first state, and
wherein, when the compartment system engages at least a portion of the foot and is adjusted from the first shape to the second shape, a position of the foot relative to the shoe is adjusted.

2. The foot orthotic system of claim 1, wherein the compartment system comprises:
a first line extending from at least one of (a) a medial edge of the shoe at a toe area of the shoe, and (b) a lateral edge of the shoe at the toe area, to at least one of (a) the medial edge at a heel area of the shoe, and (b) the lateral edge at the heel area, the medial edge extending from an end of the shoe at the toe area to an end of the shoe at the heel area, the lateral edge extending from the end of the shoe at the toe area to the end of the shoe at the heel area;
a second line extending from the medial edge to the lateral edge, the second line being substantially perpendicular to a longitudinal axis of the shoe, the longitudinal axis of the shoe extending from the end of the shoe at the toe area to the end of the shoe at the heel area;
a third line extending from the medial edge to the lateral edge, the third line being substantially perpendicular to the longitudinal axis of the shoe and disposed between the second line and the heel area;
a fourth line extending from the medial edge to the lateral edge, the fourth line being substantially perpendicular to the longitudinal axis of the shoe and disposed between the third line and the heel area;
a first medial compartment being adjustable from an unexpanded shape to an expanded shape, the first medial compartment having a foot engaging portion that is substantially bounded by the first line, the second line, the third line, and the medial edge, when the first medial compartment is in the expanded shape;
a first lateral compartment being adjustable from an unexpanded shape to an expanded shape, the first lateral compartment having a foot engaging portion that is substantially bounded by the first line, the second line, the third line, and the lateral edge, when the first lateral compartment is in the expanded shape;
a second medial compartment being adjustable from an unexpanded shape to an expanded shape, the second medial compartment having a foot engaging portion that is substantially bounded by the first line, the third line, the fourth line, and the medial edge, when the second medial compartment is in the expanded shape;
a second lateral compartment being adjustable from an unexpanded shape to an expanded shape, the second lateral compartment having a foot engaging portion that is substantially bounded by the first line, the third line, the fourth line, and the lateral edge, when the second lateral compartment is in the expanded shape;
a third medial compartment being adjustable from an unexpanded shape to an expanded shape, the third medial compartment having a foot engaging portion that is substantially bounded by the first line, the fourth line, and at least one of the medial edge at the heel area and the lateral edge at the heel area, when the third medial compartment is in the expanded shape; and
a third lateral compartment being adjustable from an unexpanded shape to an expanded shape, the third lateral compartment having a foot engaging portion that is substantially bounded by the first line, the fourth line, and at least one of the lateral edge at the heel area and the medial edge at the heel area, when the third lateral compartment is in the expanded shape.

3. The foot orthotic system of claim 1, wherein the one or more elongate members form a tether network having a cross-sectional pattern comprising at least one of a honeycomb pattern, a cobweb pattern, a mesh pattern, and a checkered pattern.

4. A method for affecting a position of a foot, the method comprising:
contacting, with a foot, a compartment system that is engaged with a shoe, the compartment system being adjustable from a first shape to a second shape; and
adjusting the compartment system from the first shape to the second shape such that a tether network, having a first section coupled to a first part of the compartment system and a second section coupled to a second part of the compartment system, changes from a first state to a second state that is more taut than is the first state,
wherein adjusting the compartment system results in a change of a position of the foot relative to the shoe.

5. The method of claim 4, wherein the adjusting the compartment system comprises inflating the compartment system with at least one of a gas and a liquid.

6. A foot orthotic system for affecting a position of a foot, the system comprising:
a compartment system configured to engage a shoe and engage at least a portion of a foot when the foot resides in or on the shoe, the compartment system being adjustable from a first shape to a second shape;
wherein, when the compartment system engages at least a portion of the foot and is adjusted from the first shape to the second shape, a position of the foot relative to the shoe is adjusted,
wherein the compartment system comprises:
a first line extending from at least one of (a) a medial edge of the shoe at a toe area of the shoe, and (b) a lateral edge of the shoe at the toe area, to at least one of (a) the medial edge at a heel area of the shoe, and (b) the lateral edge at the heel area, the medial edge extending from an end of the shoe at the toe area to an end of the shoe at the heel area, the lateral edge extending from the end of the shoe at the toe area to the end of the shoe at the heel area;
a second line extending from the medial edge to the lateral edge, the second line being substantially perpendicular to a longitudinal axis of the shoe, the longitudinal axis of the shoe extending from the end of the shoe at the toe area to the end of the shoe at the heel area;
a third line extending from the medial edge to the lateral edge, the third line being substantially perpendicular to the longitudinal axis of the shoe and disposed between the second line and the heel area;
a fourth line extending from the medial edge to the lateral edge, the fourth line being substantially perpendicular to the longitudinal axis of the shoe and disposed between the third line and the heel area;
a first medial compartment being adjustable from an unexpanded shape to an expanded shape, the first medial compartment having a foot engaging portion that is substantially bounded by the first line, the second line, the third line, and the medial edge, when the first medial compartment is in the expanded shape;
a first lateral compartment being adjustable from an unexpanded shape to an expanded shape, the first lateral compartment having a foot engaging portion that is substantially bounded by the first line, the second line, the third line, and the lateral edge, when the first lateral compartment is in the expanded shape;
a second medial compartment being adjustable from an unexpanded shape to an expanded shape, the second medial compartment having a foot engaging portion that is substantially bounded by the first line, the third line, the fourth line, and the medial edge, when the second medial compartment is in the expanded shape;
a second lateral compartment being adjustable from an unexpanded shape to an expanded shape, the second lateral compartment having a foot engaging portion that is substantially bounded by the first line, the third line, the fourth line, and the lateral edge, when the second lateral compartment is in the expanded shape;
a third medial compartment being adjustable from an unexpanded shape to an expanded shape, the third medial compartment having a foot engaging portion that is substantially bounded by the first line, the fourth line, and at least one of the medial edge at the heel area and the lateral edge at the heel area, when the third medial compartment is in the expanded shape; and
a third lateral compartment being adjustable from an unexpanded shape to an expanded shape, the third lateral compartment having a foot engaging portion that is substantially bounded by the first line, the fourth line, and at least one of the lateral edge at the heel area and the medial edge at the heel area, when the third lateral compartment is in the expanded shape,
wherein at least one of the first medial compartment, the first lateral compartment, the second medial compartment, the second lateral compartment, the third medial compartment, and the third lateral compartment comprises an inner wall and an outer wall, the inner wall and the outer wall being substantially parallel to the longitudinal axis of the shoe, and
wherein when the at least one of the first medial compartment, the first lateral compartment, the second medial compartment, the second lateral compartment, the third medial compartment, and the third lateral compartment is in the expanded shape, an average height of the outer wall is greater than an average height of the inner wall such that an angle of elevation from the inner wall at its average height to the outer wall at its average height is at least about 26 degrees.

7. The foot orthotic system of claim 6, wherein, as measured along a length of the shoe, a distance between the end of the shoe at the toe area and the second line, a distance between the second line and the third line, a distance between the third line and the fourth line, and a distance between the fourth line and the end of the shoe at the heel area are substantially equal.

8. The foot orthotic system of claim 6, wherein the second line is spaced such that between about 20% and about 35% of a length of the shoe lies between the end of the shoe at the toe area and the second line.

9. The foot orthotic system of claim 6, wherein the third line is spaced such that between about 45% and about 55% of a length of the shoe lies between the end of the shoe at the toe area and the third line.

10. The foot orthotic system of claim 6, wherein the fourth line is spaced such that between about 70% and about 85% of a length of the shoe lies between the end of the shoe at the toe area and the fourth line.

11. The foot orthotic system of claim 6, wherein the compartment system further comprises:
a fourth medial compartment being adjustable from an unexpanded shape to an expanded shape, the fourth medial compartment having a foot engaging portion that is substantially bounded by the first line, the second line, and at least one of the medial edge at the toe area and the lateral edge at the toe area, when the fourth medial compartment is in the expanded shape; and
a fourth lateral compartment being adjustable from an unexpanded shape to an expanded shape, the fourth lateral compartment having a foot engaging portion that is substantially bounded by the first line, the second line, and at least one of the lateral edge at the toe area and the medial edge at the toe area, when the fourth lateral compartment is in the expanded shape.

12. The foot orthotic system of claim 6,
wherein the first medial compartment and the first lateral compartment each comprise an inner wall and an outer wall, the inner wall and the outer wall being substantially parallel to the longitudinal axis of the shoe, and
wherein when the first medial compartment and the first lateral compartment are in their respective expanded shapes, an average height of the outer wall of the first lateral compartment is greater than an average height of the outer wall of the first medial compartment such that an angle of elevation from the outer wall of the first medial compartment at its average height to the outer wall of the first lateral compartment at its average height is at least about 17 degrees.

13. The foot orthotic system of claim 6, wherein at least a portion of the compartment system is further configured to be inflatable with at least one of a gas and a liquid.

14. The foot orthotic system of claim 6 further comprising:
one or more elongate members, each of the one or more elongate members having a first portion, a second portion, and a body therebetween, the first portion coupled to at least one of a first part of the compartment system and a first part of one of the one or more elongate members, the second portion coupled to at least one of a second part of the compartment system and a second part of one of the one or more elongate members,
wherein, as the compartment system is adjusted from the first shape to the second shape, at least one of the one or more elongate members changes from a first state to a second state that is more taut than is the first state,

15. The foot orthotic system of claim 14, wherein the first state is not taut.

16. The foot orthotic system of claim 14, wherein the first and second parts of the one of the one or more elongate members are parts of different elongate members.

17. The foot orthotic system of claim 14, wherein the one or more elongate members comprise at least one of para-aramid synthetic fiber, nylon, polyethylene, polypropylene, polystyrene, polyvinylchloride, polymethylmethacrylate, and polymethylacrylate.

18. The foot orthotic system of claim 14, wherein the one or more elongate members are disposed within an interior of the compartment system.

19. The foot orthotic system of claim 14, wherein the one or more elongate members form a tether network having a cross-sectional pattern comprising at least one of a honeycomb pattern, a cobweb pattern, a mesh pattern, and a checkered pattern.

20. The foot orthotic system of claim 6 further comprising the shoe.
